(19) United States
(12) Patent Application Publication
(10) Pub. No.: US 2022/0331574 A1
(43) Pub. Date: Oct. 20, 2022

(54) Multi-Layer Self-Healing Membrane for Septal Closure of a Medical Device

(71) Applicant: Becton, Dickinson and Company, Franklin Lakes, NJ (US)

(72) Inventors: Zehra Sevinc, Round Lake, IL (US); Michael Bailey, Creedmoor, NC (US)

(21) Appl. No.: 17/722,884

(22) Filed: Apr. 18, 2022

Related U.S. Application Data
(60) Provisional application No. 63/176,421, filed on Apr. 19, 2021.

(51) Int. Cl. A61M 39/10

(52) U.S. Cl. CPC A61M 39/10; A61M 2039/1072

(57) ABSTRACT

A membrane for a medical device includes a first portion having a first material, a second portion having a second material, where the first material is an ionomer, and where the second material is at least one of a thermoplastic elastomer, a thermoplastic polyurethane, and a thermoplastic olefin.

### CROSS-REFERENCE TO RELATED APPLICATION

The present application claims priority to U.S. Provisional Application No. 63/176,421, filed Apr. 19, 2021, entitled “Multi-Layer Self-Healing Membrane for Septal Closure of a Medical Device”, the entire disclosure of which is hereby incorporated by reference in its entirety.

### BACKGROUND OF THE INVENTION

### Field of the Disclosure

The present disclosure relates generally to a membrane for a medical device.

### Description of the Related Art

Health care providers reconstituting, transporting, and administering hazardous drugs, such as cancer treatments, can put health care providers at risk of exposure to these medications and present a hazard in the health care environment. Unintentional chemotherapy exposure can affect the nervous system, impair the reproductive system, and bring an increased risk of developing blood cancers in the future. Some drugs must be dissolved or diluted before they are administered, which involves transferring a solvent from one container to a sealed vial containing the drug in powder or liquid form, by means of a needle. Drugs may be inadvertently released into the atmosphere in gas form or by way of aerosolization, during the withdrawal of the needle from the vial and while the needle is inside the vial if any pressure differential between the interior of the vial and the surrounding atmosphere exists. In order to reduce the risk of health care providers being exposed to toxic drugs, the transfer of these drugs is accomplished utilizing a closed system transfer device or system.

Closed system transfer devices or systems may utilize membranes to ensure the safe transfer of fluid between components. For example, a syringe adapter may include a membrane that contacts a membrane of a mating component, such as a patient connector, IV bag spike, or vial adapter.

### SUMMARY OF THE INVENTION

In one aspect or embodiment, a membrane for a medical device includes a first portion having a first material, and a second portion having a second material. The first material includes an ionomer, and the second material includes at least one of a thermoplastic elastomer, a thermoplastic polyurethane, and a thermoplastic olefin.

At least one of the first material and the second material may include a lubricating additive. The lubricating additive may include at least one of boron nitride, polytetrafluoroethylene, wax, and mineral oil. The lubricating additive may be 10 wt % or less of the first portion and second portion.

The first material may have a Shore D hardness of 39-68. The first material may have a tensile strength of 2.1-5.4 kpsi. The first material may be an ethylene acrylic acid copolymer. The second material may include at least one of a styrene-based thermoplastic elastomer and an olefin-based thermoplastic elastomer. The first portion may be 25% or less of the volume of the membrane. The first portion may be an outer layer enclosing the second portion. The first portion and the second portion may be formed separately. The first portion and the second portion may be formed integrally.

In a further aspect or embodiment, a patient connector includes a body having a first end and a second end, with the body defining a passageway, a line connection positioned at the second end of the body, and a membrane according to any of the aspects or embodiments discussed above. The membrane is positioned at the first end of the body.

The membrane may be received by an opening defined by the body.

### BRIEF DESCRIPTION OF THE DRAWINGS

The above-mentioned and other features and advantages of this disclosure, and the manner of attaining them, will become more apparent and the disclosure itself will be better understood by reference to the following descriptions of aspects of the disclosure taken in conjunction with the accompanying drawings, wherein:

FIG. 1 is a front view of a medical device according to one aspect or embodiment of the present application.

FIG. 2 is a cross-sectional view of the medical device of FIG. 1.

FIG. 3 is a cross-sectional view of a membrane of the medical device of FIG. 1, according to a further aspect or embodiment of the present application.

FIG. 4 is a cross-sectional view of a membrane of the medical device of FIG. 1, according to a further aspect or embodiment of the present application.

Corresponding reference characters indicate corresponding parts throughout the several views. The exemplifications set out herein illustrate exemplary aspects of the disclosure, and such exemplifications are not to be construed as limiting the scope of the disclosure in any manner.

### DETAILED DESCRIPTION

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

Unless otherwise indicated, all ranges or ratios disclosed herein are to be understood to encompass the beginning and ending values and any and all subranges or subratios subsumed therein. For example, a stated range or ratio of “1 to 10” should be considered to include any and all subranges or subratios between (and inclusive of) the minimum value of 1 and the maximum value of 10; that is, all subranges or subratios beginning with a minimum value of 1 or more and ending with a maximum value of 10 or less.

The terms “first”, “second”, and the like are not intended to refer to any particular order or chronology, but refer to different conditions, properties, or elements.

As used herein, “at least one of” is synonymous with “one or more of”. For example, the phrase “at least one of A, B, and C” means any one of A, B, or C, or any combination of any two or more of A, B, or C. For example, “at least one of A, B, and C” includes one or more of A alone; or one or more of B alone; or one or more of C alone; or one or more of A and one or more of B; or one or more of A and one or more of C; or one or more of B and one or more of C; or one or more of all of A, B, and C.

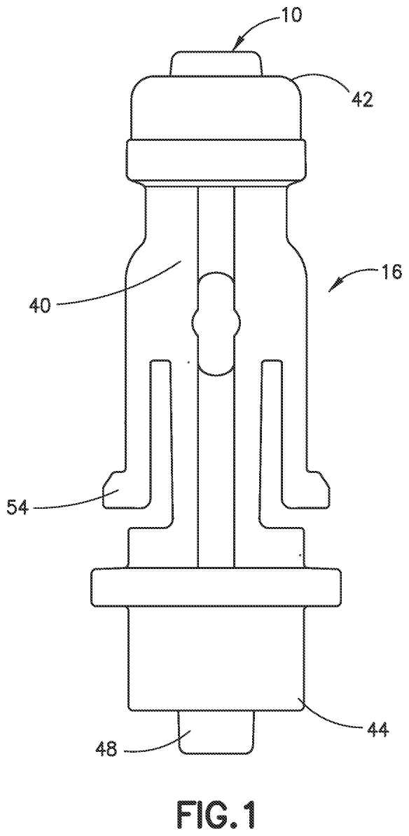

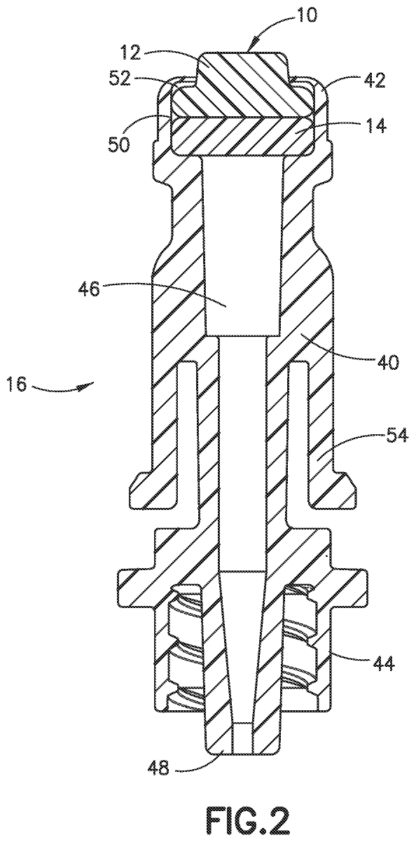

Referring to FIGS. 1-4, a membrane 10 for a medical device according to one aspect or embodiment of the present application includes a first portion 12 having a first material and second portion 14 having a second material. As shown in FIGS. 1 and 2, the membrane 10 is shown in connection with a medical device 16. More specifically, the medical device 16 is a patient connector, which is utilized to connect one component of a closed system transfer device or system to a patient intravenous line. For example, the medical device 16 may be connected to a syringe adapter to facilitate the transfer of fluid from one container, such as a syringe barrel, to another container or line, such as an intravenous line, IV bag, or other component. The membrane 10 may be utilized in any component of a closed system transfer device or system. In one aspect or embodiment, the membrane 10 is utilized in each component that mates with a syringe adapter, such as a patient connector, vial adapter, IV bag spike, etc. The syringe adapter may be the same and operate in the same manner as the syringe adapter shown and described in United States Patent Application Publication No. 2015/0297454, which is hereby incorporated by reference in its entirety.

Referring to FIGS. 1 and 2, the first material of the first portion 12 of the membrane 10 is an ionomer. In one aspect or embodiment, the first material is an ethylene acrylic acid copolymer. The first material is configured to be self-healing. The first material is also configured to be abrasion and puncture resistant. In one aspect or embodiment, the first material is a SURLYN™ series ionomer commercially available from Dow Inc.

In one aspect or embodiment, the first material has the following properties:

TABLE 1

| Property | Value Range | Test Method |
|---|---|---|
| Specific Gravity | 0.94-0.97 | ASTM D 792 |
| Shore D Hardness | 39-68 | ASTM D2240 |
| Flex Modulus (room | 4.3-75 | ASTM D790 |
| temperature, kpsi) | | |
| Tensile Strength (kpsi) | 2.1-5.4 | ASTM D 638 |
| Elongation at Break (%) | 285-660 | ASTM D 638 |

In one aspect or embodiment, the second material of the second portion 14 of the membrane 10 is at least one of a thermoplastic elastomer, a thermoplastic polyurethane, and a thermoplastic olefin. In one aspect or embodiment, the second material is at least one of a styrene-based thermoplastic elastomer and an olefin-based thermoplastic elastomer. Forming the membrane 10 with the first portion 12 with the first material and the second portion 14 with the second material provides a membrane that is configured to minimize fragmentation due to the self-healing property of the first portion 12 while still providing acceptable hardness, compression set, insertion and retraction force of a needle moving through the membrane 10, and cost due to the properties of the second portion 14.

In one aspect or embodiment, the first material of the first portion 12 is an acid copolymer, such as copolymer of ethylene and methacrylic acid. More specifically, in one aspect or embodiment, the first material of the first portion 12 may be NUCREL™ acid copolymer commercially available from Dow Inc. Commercial grades of ethylene (meth)acrylic acid (EAA, EMAA) are available from DuPont (Surlyn™, Nucrel™), SK Global Chemical (Primacor™), and Ineos (Eltex®).

In further aspects or embodiments, the first material of the first portion 12 may be a blended material with other polymers for property optimization such as blends of 15 to 50 wt % ionomers within at least one of ethylene-vinyl acetate (EVA), thermoplastic elastomer (TPE), thermoplastic olefin (TPO), and thermoplastic polyurethane (TPU).

In another aspect or embodiment, the first material of the first portion 12 is Hycar® butadiene-acrylic acid commercially available from Lubrizol, Amplify™ ethylene-acrylic acid commercially available from Dow Inc.

In another aspect or embodiment, the first material of the first portion 12 is at least one of phosphonated ethylene propylene diene monomer rubber EPDM, phosphonated polyethylene, lightly sulfonated polystyrene (SPS), and poly(styrene-co-methyl acrylate).

In one aspect or embodiment, the second material of the second portion 14 is at least one of Dynaflex™ TPE commercially available from Avient, Versaflex™ TPE commercially available from Avient, and Medalist® MD-12340 TPE commercially available from Teknor.

In one aspect or embodiment, the second material has the following properties:

TABLE 2

| Property | Value Range | | Test Method |
|---|---|---|---|
| Shore A Hardness | 43 | | ASTM D2240 |
| Tensile Stress (100% | 180 | psi | ASTM D412 |
| strain; 73° F.) | | | |
| Tensile Strength | 830 | psi | ASTM D412 |
| (Break; 73° F.) | | | |
| Tear Strength | 130 | lbf/in. | ASTM D624 |

| | | |
|---|---|---|
| Compression Set | 14% | ASTM D395B |
| (73° F.; 22.0 hr) | | |

In one aspect or embodiment, the first portion 12 and the second portion 14 are formed separately. The first portion 12 and the second portion 14 may also be formed integrally. In one aspect or embodiment, the membrane 10 is formed using a co-injection molding process where the first and second materials are injected simultaneously into the mold. In a further aspect or embodiment, the membrane 10 is formed using an injection-compression molding process including an injection phase and a compression phase.

In one aspect or embodiment, the first material and/or the second material includes a lubricating additive. The lubricating additive may be at least one of boron nitride, polytetrafluoroethylene, wax, and mineral oil, although other suitable lubricating additives may be utilized. In one aspect or embodiment, the lubricating additive is 10 wt % or less of the first portion 12 and the second portion 14.

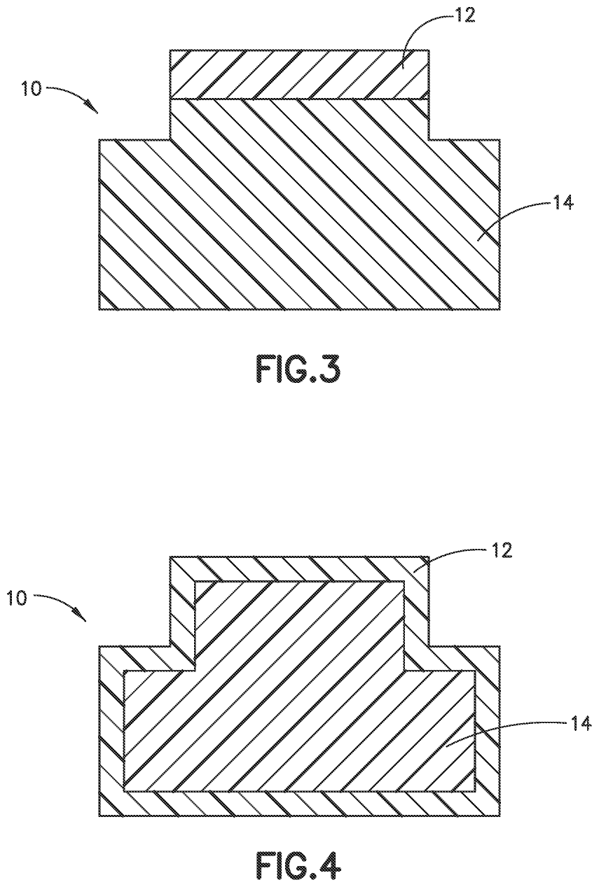

Referring to FIG. 3, in one aspect or embodiment, the first portion 12 of the membrane 10 is thinner than the second portion 14 of the membrane. The first portion 12 may be 25% or less, 10% or less, 5% or less, or 1% or less of the volume of the membrane 10. The first portion 12 may also be 10% or less of the volume of the membrane 10. The first portion 12 of the membrane 10 is configured to engage a corresponding membrane of a mating connector.

Referring to FIG. 4, in one aspect or embodiment, the first portion 12 of the membrane 10 is an outer layer enclosing the second portion 14 of the membrane 10. The first portion 12 may entirely cover an outer surface of the second portion 14 of the membrane 10.

In one aspect or embodiment, the first material and/or second material of the first and second portions 12,14 may include at least one of an antioxidant, UV absorber, energy quencher, and pigment for coloring. These additives may be less than 1 wt % of the membrane 10.

In one aspect or embodiment, the membrane 10 has a thickness of 5.5 mm with the first portion 12 having a minimum thickness of 0.76 mm up to a maximum of 3.17 mm.

Referring again to FIGS. 1 and 2, the medical device 16 includes a body 40 having a first end 42 and a second end 44, with the body 40 defining a passageway 46, a line connection 48 positioned at the second end 44 of the body 40, and the membrane 10 positioned at the first end 42 of the body 40. The line connection 48 may be a luer lock connection, although other suitable connections may be utilized. The membrane 10 is received by an opening 50 defined by the body 40 of the patient connector 16. The body 40 of the patient connector 16 includes a securing extension 52 at the first end 42 of the body 40, with the securing extension 52 extending radially inward and configured to secure the membrane 10 to the body 40 of the patient connector 16. The patient connector 16 also includes a locking arrangement 54 configured to secure the patient connector 16 to a syringe adapter.

While this disclosure has been described as having exemplary designs, the present disclosure can be further modified within the spirit and scope of this disclosure. This application is therefore intended to cover any variations, uses, or adaptations of the disclosure using its general principles. Further, this application is intended to cover such departures from the present disclosure as come within known or customary practice in the art to which this disclosure pertains and which fall within the limits of the appended claims. To the extent possible, one or more features of any aspect or embodiment discussed above can be combined with one or more features of any other aspect or embodiment.

What is claimed is:

1. A membrane for a medical device comprising:
a first portion comprising a first material; and
a second portion comprising a second material, wherein the first material comprises an ionomer, and wherein the second material comprises at least one of a thermoplastic elastomer, a thermoplastic polyurethane, and a thermoplastic olefin.

2. The membrane of claim 1, wherein at least one of the first material and the second material comprise a lubricating additive.

3. The membrane of claim 2, wherein the lubricating additive comprises at least one of boron nitride, polytetrafluoroethylene, wax, and mineral oil.

4. The membrane of claim 3, wherein the lubricating additive is 10 wt % or less of the first portion and the second portion.

5. The membrane of claim 1, wherein the first material has a Shore D hardness of 39-68.

6. The membrane of claim 1, wherein the first material has a tensile strength of 2.1-5.4 kpsi.

7. The membrane of claim 1, wherein the first material comprises an ethylene acrylic acid copolymer.

8. The membrane of claim 1, wherein the second material comprises at least one of a styrene-based thermoplastic elastomer and an olefin-based thermoplastic elastomer.

9. The membrane of claim 1, wherein the first portion comprises 25% or less of the volume of the membrane.

10. The membrane of claim 1, wherein the first portion is an outer layer enclosing the second portion.

11. The membrane of claim 1, wherein the first portion and the second portion are formed separately.

12. The membrane of claim 1, wherein the first portion and the second portion are formed integrally.

13. A patient connector comprising:
a body having a first end and a second end, the body defining a passageway;
a line connection positioned at the second end of the body; and
a membrane positioned at the first end of the body, the membrane comprising:
a first portion comprising a first material, and
a second portion comprising a second material, wherein the first material comprises an ionomer, and wherein the second material comprises at least one of a thermoplastic elastomer, a thermoplastic polyurethane, and a thermoplastic olefin.

14. The patient connector of claim 13, wherein the membrane is received by an opening defined by the body.

15. The patient connector of claim 13, wherein at least one of the first material and the second material of the membrane comprise a lubricating additive comprising at least one of boron nitride, polytetrafluoroethylene, wax, and/or mineral oil.

16. The patient connector of claim 13, wherein the first material of the membrane has a Shore D hardness of 39-68.

17. The patient connector of claim 13, wherein the first material of the membrane comprises an ethylene acrylic acid copolymer.

18. The patient connector of claim 13, wherein the second material of the membrane comprises at least one of a styrene-based thermoplastic elastomer and an olefin-based thermoplastic elastomer.

19. The patient connector of claim 13, wherein the first portion of the membrane comprises 25% or less of the volume of the membrane.

20. The patient connector of claim 13, wherein the first portion of the membrane is an outer layer enclosing the second portion.
